(19) United States
(12) Patent Application Publication
(10) Pub. No.: US 2007/0116678 A1
(43) Pub. Date: May 24, 2007

(54) Medical device with living cell sheet

(76) Inventors: Hsing-Wen Sung, HsinChu (TW); Mei-Chin Chen, Taipei County (TW); Hosheng Tu, Newport Beach, CA (US)

Correspondence Address: HOSHENG TU, 15 RIEZ, NEWPORT BEACH, CA, 92657-0116, US

(21) Appl. No.: 11/412,610

(22) Filed: Apr. 27, 2006

Related U.S. Application Data
(63) Continuation-in-part of application No. 11/287,541, filed on Nov. 23, 2005.

(51) Int. Cl. A61K 35/12

(52) U.S. Cl. 424/93.7

(57) ABSTRACT

A novel method, using a thermoreversible MC/PBS/Collagen hydrogel coated on the TCPS dish, for harvesting a living cell sheet with ECM. The obtained living cell sheet is administered to a joint adapted for implantation and for cartilage regeneration.

### CROSS-REFERENCE TO RELATED APPLICATIONS

This application is a continuation-in-part application of U.S. patent application Ser. No. 11/287,541, filed Nov. 23, 2005, entitled “Living Cell Sheet”, the entire contents of which are incorporated herein by reference.

### FIELD OF THE INVENTION

The present invention is related to living cell sheets for tissue reconstructions and regeneration, more particularly; the invention is related to a medical device having a sheet derived from a thermoreversible hydrogel for harvesting living cells.

### BACKGROUND OF THE INVENTION

Fetal cardiomyocytes or stem cells transplanted into myocardial scar tissue improved heart function. However, low cell numbers remain in place because of washout effects. The transplanted allogenic cells survive for only a short time in the recipient heart because of immunorejection. Autologous cell transplantation would be ideal. The cultured skeletal myoblasts have been successfully isolated, cultured, and transplanted into injured and normal myocardium of the same animal. One of the basic problems with cell therapy in myocardial infarct patients is cell leakage from the implanted site.

Methylcellulose (MC) is a water-soluble polymer derived from cellulose, the most abundant polymer in nature. As a viscosity-enhancing polymer, it thickens a solution without precipitation over a wide pH range. This feature makes it widely useable as a thickener in the food and paint industries. It is recognized as an acceptable food additive by the U.S. Food and Drug Administration. Additionally, the physiological inertness and the storage stability of MC permit its use in cosmetics and pharmaceutical products.

Recently, investigations of hydrogels have focused on functional hydrogels. These functional hydrogels may change their structures as they expose to varying environment, such as temperature, pH, or pressure. MC becomes gels from aqueous solutions upon heating or salt addition (Langmuir 2002; 18:7291, Langmuir 2004; 20:6134). This unique phase-transition behavior of MC makes it as a promising functional hydrogel for various biomedical applications (Biomaterials 2001; 22:1113, Biomacromolecules 2004; 5:1917). Tate et al. studied the use of MC as a thermoresponsive scaffolding material (Biomaterials 2001; 22:1113). In their study, MC solutions were produced to reveal a low viscosity at room temperature and formed a soft gel at 37° C.; thus making MC well suited as an injectable scaffold for the repair of defects in the brain. Additionally, using its thermoresponsive feature, MC was used by our group to harden aqueous alginate as a pH-sensitive based system for the delivery of protein drugs (Biomacromolecules 2004; 5:1917).

It is disclosed herein that a novel application of this thermoresponsive MC hydrogel is blended with distinct salts and coated on tissue culture polystyrene (TCPS) dishes as a living-cell-sheet harvest system. It was reported that a thermoresponsive polymer, poly(N-isopropylacrylamide) (PNIPAAm), is chemically grafted on TCPS dishes to develop a cell-sheet for tissue reconstructions (J. Biomed. Mater. Res. 1993; 27:1243). PNIPAAm is hydrophobic at 37° C. and hydrophilic at 20° C., thus the cultured cells can be harvested as a continuous cell sheet after incubation at 20° C. The harvested cell sheets have been used for various tissue reconstructions, including ocular surfaces, periodontal ligaments, cardiac patches, and bladder augmentations (Materials today 2004; 42). In their method, PNIPAAm is polymerized and concurrently grafted to TCPS dishes by means of irradiation with an electron beam. The whole grafting process is relatively complicated and time-consuming (Tissue Eng. 2005; 11:30).

It is herein disclosed that a simple and inexpensive method is provided by simply pouring aqueous MC solutions blended with distinct salts on TCPS dishes at room temperature (about 20° C.) and subsequently gelled at 37° C. (the MC hydrogel). The gelled coating at 37° C. is then evenly spread with a neutral aqueous collagen at 4° C. The spread aqueous collagen gradually reconstitutes with time and thus forms a thin layer of collagen coated on the MC hydrogel. The physical behavior of the prepared MC hydrogels transitions from the solution to a gel state as a function of temperature.

In the orthopedic field, degenerative arthritis or osteoarthritis is the most frequently encountered disease associated with cartilage damage. Almost every joint in the body, such as the knee, the hip, the shoulder, and even the wrist, is affected. The pathogenesis of this disease is the degeneration of hyaline articular cartilage. The hyaline cartilage of the joint becomes deformed, fibrillated, and eventually excavated. If the degenerated cartilage could somehow be regenerated, most patients would be able to enjoy their lives without debilitating pain.

U.S. Patent Application publication no. 2005/0074481, published on Apr. 7, 2005, entire contents of which are incorporated herein by reference, discloses an implantable device for facilitating the healing of voids in bone, cartilage and soft tissue, comprising a polyelectrolytic complex region joined with a subchondral bone region. The polyelectrolytic complex region enhances the environment for chondrocytes to grow articular cartilage; while the subchondral bone region enhances the environment for cells which migrate into that region's macrostructure and which differentiate into osteoblasts.

U.S. Patent Application publication no. 2005/0159820, published on Jul. 21, 2005, entire contents of which are incorporated herein by reference, discloses a member for articular cartilage regeneration being characterized in that the member comprises a hydroxyapatite porous element having a number of pores distributed therein, substantially all of the pores being three-dimensionally communicated to each other through open portions.

An exemplary articular cartilage repairing means that can be used in a method of the invention is described in U.S. Pat. No. 6,835,377 B2, which discloses mesenchymal stem cells for articular cartilage repair combined with a controlled-resorption biodegradable matrix, preferably collagen-based products. These mesenchymal stem cell-matrix implants initiate tissue formation, and maintain and stabilize the articular defect during the repair process. In addition to gels, the types of biomatrix materials that may be used include sponges, foams or porous fabrics that form a three-dimensional scaffold for the support of mesenchymal stem cells. These materials may be composed of collagen, gelatin, hyaluronan or derivatives thereof, may consist of synthetic polymers, or may consist of composites of several different materials. The different matrix configurations and collagen formulations will depend on the nature of the cartilage defect, and include those for both open surgical and arthroscopic procedures.

Human mesenchymal stem cell technology provides not only multiple opportunities to regenerate cartilage, but other mesenchymal tissue as well, including bone, muscle, tendon, marrow stroma and dermis. The regeneration of cartilage and other injured or diseased tissue is achieved by administration of an optimal number of human mesenchymal stem cells to the repair site in an appropriate biomatrix delivery device, without the need for a second surgical site to harvest normal tissue grafts. However, cells without a colony or confluence arrangement usually fails to sustain the proliferation and stability.

Clearly, there remains a need to develop a system and methods whereby living cells on a sheet can be delivered to a deficiency or defect site for treating bone or joint defect in a patient. In view of the foregoing, an object of this invention is to provide a novel method, using a thermoreversible MC/PBS/Collagen hydrogel coated on the TCPS dish, for harvesting a living cell sheet with ECM. The coated hydrogel system is reusable and can be used for culturing a multi-layer cell sheet. The obtained living cell sheets are useful for tissue reconstructions and cell separation.

### SUMMARY OF THE INVENTION

Some aspects of the invention relate to a novel yet simple method, using a thermoreversible hydrogel system that is coated on tissue culture polystyrene (TCPS) dishes, to provide means for harvesting living cell sheets. The hydrogel system is prepared by simply pouring aqueous methylcellulose (MC) solutions blended with distinct salts on TCPS dishes at 20° C. In one embodiment, aqueous MC compositions form a gel at 37° C. for the application of cell cultures. In one embodiment, the hydrogel coating composed of 8% MC blended with 10 g/L PBS (the MC/PBS hydrogel, with a gelation temperature of about 25° C.) stayed intact throughout the entire course of cell culture.

Some aspects of the invention relate to cell attachments comprising evenly spreading the MC/PBS hydrogel at 37° C. with a neutral aqueous collagen at 4° C. The spread aqueous collagen gradually reconstitutes with time and thus forms a thin layer of collagen (the MC/PBS/Collagen hydrogel). After cells reaching confluence, a continuous monolayer cell sheet forms on the surface of the MC/PBS/Collagen hydrogel. When the grown cell sheet is placed outside of the incubator at 20° C., it detaches gradually from the surface of the thermoreversible hydrogel spontaneously, in absence of any enzymes.

Some aspects of the invention relate to a method of preparing a living cell sheet comprising: coating a thermoreversible hydrogel on a tissue culture dish, wherein the hydrogel comprises methylcellulose, phosphate buffered saline, and optionally collagen; loading target living cells into the dish; incubating the dish for a predetermined duration; and removing the sheet from the dish.

Some aspects of the invention relate to a method of preparing a 3-D living cell construct comprising: coating a thermoreversible hydrogel on a 3-D scaffold support element, wherein the hydrogel comprises methylcellulose, phosphate buffered saline, and collagen; loading target living cells onto the support element; and incubating the support element for a predetermined duration. In one embodiment, the method further comprises a step of removing the construct from the support element.

The results obtained in the MTT assay demonstrate that the cells cultured on the surface of the MC/PBS/Collagen hydrogel had better cell activities than those cultured on an uncoated TCPS dish. After harvesting the detached cell sheet, the remained viscous hydrogel system is reusable. Additionally, the developed hydrogel system is used for culturing a multi-layer cell sheet. The obtained living cell sheets are candidates for tissue reconstructions or tissue regeneration. In one embodiment, the cells of the invention comprise mesenchymal stem cells, adult multipotent cells, progenitor cells, marrow stromal cells. In a further embodiment, the cells of the invention comprise the intermediate cells, such as osteoblast leading to bone, chondrocyte leading to cartilage, adipocyte leading to adipose, and other cell types leading to connective tissue.

Some aspects of the invention provide a composite medical device or an implant comprising a living cell sheet and a support scaffold having at least two layers, wherein the living cell sheet is sandwiched in between the two layers, wherein at least a portion of the sandwiched two layers are further secured to each other. In one embodiment, the method for securing the two layers is selected from a group consisting of sealing, coupling, stapling, and suturing. Furthermore, the living cell sheet is manufactured by a process comprising: coating a thermoreversible hydrogel on a tissue culture dish, wherein the hydrogel comprises methylcellulose, and phosphate buffered saline; loading target living cells into the dish; incubating the dish for a predetermined duration; and removing the sheet from the dish.

In one embodiment, the support scaffold is biodegradable and the living cell sheet may comprise mesenchymal stem cells. In another embodiment, the medical device or the implant may comprise a wound dressing device, a valvular leaflet, a bioprosthetic tissue valve, a ligament tendon substitute, a tendon substitute, a breast insert for breast tissue regeneration, and the like.

It is one object of the present invention to provide a manufacturing process for the support scaffold, wherein the process comprises: removing cellular material from a nature tissue, wherein porosity of the nature tissue is increased at least 5%, the increase of porosity being adapted for promoting tissue regeneration. In one embodiment, increased porosity is provided by an acellularization process, an acid treatment process, a basic treatment process, or an enzyme treatment process. In another embodiment, the manufacturing process further comprises a step of crosslinking the nature tissue.

Some aspects of the invention provide a method for treating a target tissue, comprising: providing a composite medical device comprising a living cell sheet and a support scaffold having at least two layers, wherein the living cell sheet is sandwiched in between the two layers, and wherein at least a portion of the sandwiched two layers are further secured to each other; delivering the composite medical device to the target tissue; and treating the target tissue by cell proliferation. In one embodiment, the living cell sheet comprises mesenchymal stem cells.

Some aspects of the invention provide a composite medical device that is broken up to pieces sized and configured for loading in the delivery instrument.

Some aspects of the invention provide a method for treating a target tissue, comprising: providing a living cell sheet, wherein the living cell sheet is manufactured by a process comprising coating a thermoreversible hydrogel on a tissue culture dish, wherein the hydrogel comprises methylcellulose, and phosphate buffered saline, loading target living cells into the dish, incubating the dish for a predetermined duration, and removing the sheet from the dish; delivering the living cell sheet to the target tissue; and treating the target tissue by cell proliferation. In one embodiment, the living cell sheet is cut, sized, and configured for loading inside a delivery instrument. In another embodiment, the living cell sheet is a strip sheet that is appropriately loaded inside the lumen of the delivery instrument.

Some aspects of the invention provide a method for treating a joint defect in an animal, comprising administering to the animal stem cells, the stem cells being configured in a living cell sheet. In one embodiment, the living cell sheet is sized and configured to be planar at about 100 microns in size (i.e., equivalent diameter) and about one cell thickness. In another embodiment, the living cell sheet contains about at least 100 cells.

Some aspects of the invention provide a method for treating cartilage defects in a patient, comprising delivering to the patient human cells in a sheet form, wherein the human cell sheet covers or contacts at least a portion of the defects, wherein the human cells are mesenchymal stem cells, marrow stromal cells, or chondrocytes.

### BRIEF DESCRIPTION OF THE DRAWINGS

FIG. 1 shows the DSC thermograms of aqueous methylcellulose solutions (2% by w/v) blended with distinct concentrations of NaCl.

FIG. 2 shows gelation temperatures of aqueous methylcellulose solutions blended with distinct salts: effect of the concentration of salt.

FIG. 3 shows gelation temperatures of aqueous methylcellulose solutions blended with distinct salts: effect of the concentration of methylcellulose.

FIG. 4 shows osmolalities of aqueous methylcellulose solutions blended with distinct salts: effect of the concentration of salt.

FIG. 5 shows osmolalities of aqueous methylcellulose solutions blended with distinct salts: effect of the concentration of methylcellulose.

FIG. 6 shows changes in osmolality of the PBS solution loaded on each studied TCPS dish with time.

FIG. 7 shows photographs of the TCPS dish coated with the MC/PBS hydrogel in sequence: (a) at 20° C.; (b) at 37° C. for 5 min; (c) at 37° C. for 30 min; (d) followed by at 20° C. for 2 min; and (e) followed by at 20° C. for 20 min.

FIG. 8 shows photomicrographs of cells cultured on: (a) an uncoated TCPS dish, 40×; (b) the TCPS dish coated with the 2% MC+1M NaCl hydrogel, 40×; (c) the TCPS dish coated with the 2% MC+0.2M Na₂SO₄ hydrogel, 40×; (d) the TCPS dish coated with the 2% MC+0.2M Na₃PO₄ hydrogel, 40×; and (e) the TCPS dish coated with the MC/PBS (8% MC+10 g/L PBS) hydrogel, 40× and (f) 100×.

FIG. 9 shows schematic illustrations of cells cultured on the TCPS dish coated with the MC/PBS/Collagen hydrogel and detachment of its grown cell sheet.

FIG. 10 shows photomicrographs of cells cultured on: (a) an uncoated TCPS dish; and (b) on the TCPS dish coated with the MC/PBS/Collagen hydrogel for 1, 3, and 7 days, respectively.

FIG. 11 shows photographs of (a) a grown cell sheet on the TCPS dish coated with the MC/PBS/Collagen hydrogel, and (b) its detaching cell sheet. Photomicrographs of the detaching cell sheet with time as (c) to (j).

FIG. 12 shows immunofluorescence images of the cell sheets grown on the TCPS dish coated with the MC/PBS/Collagen hydrogel for: (a) 1 week; and (b) 2 weeks.

FIG. 13 shows immunofluorescence images of: (a) a single-layer cell sheet (CS); (b) a double-layer cell sheet; and (c) a tri-layer cell sheet obtained from the TCPS dish coated with the MC/PBS/Collagen hydrogel; and (d) a tri-layer cell sheet obtained by folding a single-layer cell sheet.

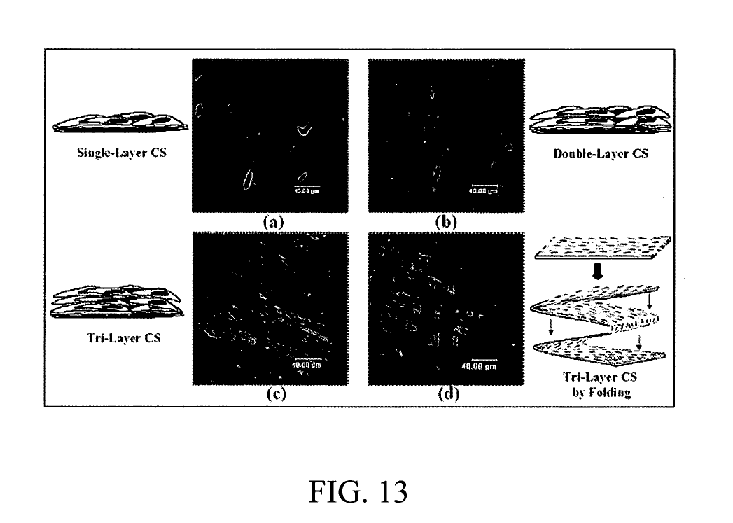

FIG. 14 shows a medical device comprising a support scaffold structure of multiple layers that sandwich a single cell sheet in between two adjacent scaffold layers.

FIG. 15 shows cell sheet preparation and injection methods.

### DETAILED DESCRIPTION OF THE EXEMPLARY EMBODIMENTS

The preferred embodiments of the present invention described below relate particularly to preparation of sheets derived from a thermoreversible hydrogel coated on a tissue culture polystyrene dish for harvesting living cells. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

By “living cell sheet” is meant herein any configuration or shape of contiguous living cells arranged and formed from living cells, wherein each living cell sheet may comprise tens or more of cells, preferably at least 100 cells, and most preferably at least one thousand cells, in a partially overlapped layers, preferably in a single layer. In one embodiment, the contiguous living cells are connected through extracellular matrix and appear confluent. The living cell sheet may be configured in a ball, a pellet, an aggregate, a cylindrical, a wrinkled sheet, or any appropriate configuration for delivery and placement at a target tissue site. In a further embodiment, the single cell sheet is sized and configured to be planar (the sheet thickness is about one cell size) about 500 microns in average sizes, preferably about 100 microns, and most preferably about 50 microns in average planar sizes.

### EXAMPLE NO. 1

### Gelation of Aqueous MC Solutions

Commercial MC is a heterogeneous polymer consisting of highly substituted zones (hydrophobic zones) and less substituted ones (hydrophilic zones). Aqueous MC solutions undergo a sol-gel reversible transition upon heating or cooling. In the solution state at lower temperatures, MC molecules are hydrated and there is little polymer-polymer interaction other than simple entanglements. As temperature is increased, aqueous MC solutions absorb energy (the endothermic peaks observed in the differential scanning calorimeter, DSC, thermograms discussed later) and gradually lose their water of hydration. Eventually, a polymer-polymer association takes place, due to hydrophobic interactions, causing cloudiness in solution and subsequently forming an infinite gel-network structure (Carbohydr. Polym. 1995; 27:177).

The temperature in forming this gel-network structure, at which the aqueous MC solution does not flow upon inversion of its container, is defined as the gelation temperature herein. Therefore, the gelation temperature of the aqueous MC solution determined by inverting its container should be slightly greater than the onset temperature of the endothermic peak observed in its corresponding DSC thermogram.

It was reported that addition of salts lowers the gelation temperature of the aqueous MC solution (Langmuir 2002; 18:7291). Upon addition of salts, water molecules are placed themselves around the salts, thus reducing the intermolecular hydrogen-bond formations between water molecules and the hydroxyl groups of MC. This can increase the hydrophobic interaction between MC molecules and lead to a decrease in their gelation temperature.

### EXAMPLE NO. 2

### Preparation of Aqueous MC Solutions

MC (with a viscosity of 3,000-5,500 cps for a 2% by w/v aqueous solution at 20° C.) was obtained from Fluka (64630 Methocel® MC, Buchs, Switzerland). Aqueous MC solutions in different concentrations (1%, 2%, 3%, or 4% by w/v) were prepared by dispersing the weighed MC powders in heated water with the addition of distinct salts (NaCl, Na₂SO₄, Na₃PO₄) or in phosphate buffered saline (PBS) in varying concentrations at 50° C. The osmolalities of the prepared aqueous MC solutions were then measured using an osmometer (Model 3300, Advanced Instruments, Inc., Norwood, Mass., USA).

### EXAMPLE NO. 3

### Gelation Temperatures of Aqueous MC Solutions

The physical gelation phenomena of aqueous MC solutions with temperature were visually observed and measured by a DSC (Pyris Diamond, Perkin Elmer, Shelton, Conn., USA). Aqueous MC solutions blended with distinct salts (2 ml samples) were exposed to elevating temperatures via a standard hot-water bath. Behavior was recorded at intervals of approximately 0.5° C. over the range of 20-70° C. The heating rate between measurements was approximately 0.5° C./min. At each temperature interval, the solutions/gels were allowed to equilibrate for 30 min. A “gel” criterion was defined as the temperature at which the solution did not flow upon inversion of the container. A DSC was used to determine the transition temperatures of the prepared aqueous MC solutions heating from 20 to 90° C. A heating rate of 10° C./min was used for all test samples.

### EXAMPLE NO. 4

### Preparation of the MC-Hydrogel Coated TCPS Dish

The prepared aqueous MC solutions that had a gelation temperature below 37° C. were used to coat TCPS dishes (Falcon® 3653, diameter 35 mm, Becton Dickinson Labware, Franklin Lakes, N.J., USA). A 450 μl of test MC solutions was poured into the center of each TCPS dish at room temperature (about 20° C.). A thin transparent layer of the poured solution was evenly distributed on the TCPS dish. Subsequently, the TCPS dish was pre-incubated at 37° C. for 1 hour and a gelled opaque layer (the MC hydrogel) was formed on the dish. To evaluate whether the salts blended in the MC hydrogel would leach out with time, the coated TCPS dish was loaded with a pre-warmed PBS at 37° C. (2 ml, with an osmolality of 280±10 mOsm/kg). The osmolality of the loaded PBS solution was monitored with time. An uncoated TCPS dish loaded with the same PBS was used as a control.

For the system further coated with collagen, a 0.5 mg/ml aqueous type I collagen (bovine dermis collagen, Sigma Chemical Co., St. Louis, Mo., USA), adjusted to pH 7.4 by dialysis against PBS at 4° C., was evenly spread onto the aforementioned TCPS dish coated with the MC hydrogel at 37° C.

### EXAMPLE NO. 5

### Cell Culture

HFF (human foreskin fibroblasts) were cultured in Dulbecco's modified Eagle's Minimal Essential Medium (12800 Gibco, Grand Island, N.Y., USA) supplemented with 10% fetal bovine serum (JRH, Brooklyn, Australia) and 0.25% penicillin-streptomycin (15070 Gibco, Grand Island, N.Y., USA) in the TCPS dish of Example No. 4. The cells were maintained at 37° C. with 5% CO₂ and the cultured media were changed 3 times a week until ready for use. In one embodiment, some appropriate growth factors may be added into the culture media, wherein the growth factor may be selected from the group consisting of VEGF (vascular endothelial growth factor), VEGF 2, bFGF (basic fibroblast growth factor), aFGF (acidic fibroblast growth factor), VEGF121, VEGF165, VEGF189, VEGF206, PDGF (platelet derived growth factor), PDAF (platelet derived angiogenesis factor), TGF-β (transforming growth factor-β1, β2, β3 and the like), PDEGF (platelet derived epithelial growth factor), PDWHF (platelet derived wound healing factor), insulin-like growth factor, epidermal growth factor, hepatocytic growth factor, and combinations thereof. After reaching confluence, cells were isolated from culture dishes with a 0.05% trypsin and then seeded uniformly on the coated TCPS dishes at a density of 4×10⁴ cells/cm² at 37° C. Cell attachment and growth were observed daily using a microscope. An uncoated TCPS dish was used as a control. Cell viability was assessed by the MTT [3-(4,5-dimethylthiazol-yl)-2,5-diphenyltetrazolium bromide, Sigma] assay. Details of the methodology used in the MTT assay were previously described (J. Biomed. Mater. Res. 2002; 61:360).

### EXAMPLE NO. 6

### Detachment of Cell Sheets

Cells grown on the dishes for 1 or 2 weeks (with media changes 3 times per week) were taken out from the incubator with media present. The dishes were then allowed to cool at approximately 20° C. Changes in morphology of cell sheets on the dishes with time were photographed every 5 seconds for up to 15 min.

### EXAMPLE NO. 7

### Immunofluorescence Staining

Monoclonal mouse anti-collagen type I (1:150, ICN Biomedicals, Inc., Aurora, Ohio, USA) and type III (1:200, Chemicon International Inc., Temecula, Calif., USA) antibodies were used for localizing type I and type III collagen secreted by HFF, respectively. A Cy5-conjugated affinity-purified goat anti-mouse IgG+IgM (H+L) (1.5 mg/ml, Jackson ImmunoResearch Laboratories, Inc., Pa., USA) was used as the secondary antibody for labeling the monoclonal antibody. Cell sheets grown on the dishes were fixed in 4% phosphate buffered formaldehyde at 37° C. for 10 minutes and then permeabilized with 0.1% Triton X-100 in PBS containing 1% bovine serum albumin (PBS-BSA) and RNase 100 μg/ml. After washing 3 times with PBS-BSA, the cell sheets were exposed to the primary antibody for 60 min at 37° C. The cell sheets were then incubated for another 60 min with the secondary antibody (1:400) at room temperature. Additionally, the cell sheets were co-stained to visualize F-actins and nuclei acids by phalloidin (Oregon Green® 514 phalloidin, Molecular Probes, Inc., Eugene, Oreg., USA) and propidium iodide (PI, P4864, Sigma), respectively.

Subsequently, the stained cell sheets were evenly mounted on the slides and examined with excitations at 488, 543, and 633 nm, respectively, using an inversed confocal laser scanning microscope (TCS SL, Leica, Germany). Superimposed images were performed with an LCS Lite software (version 2.0).

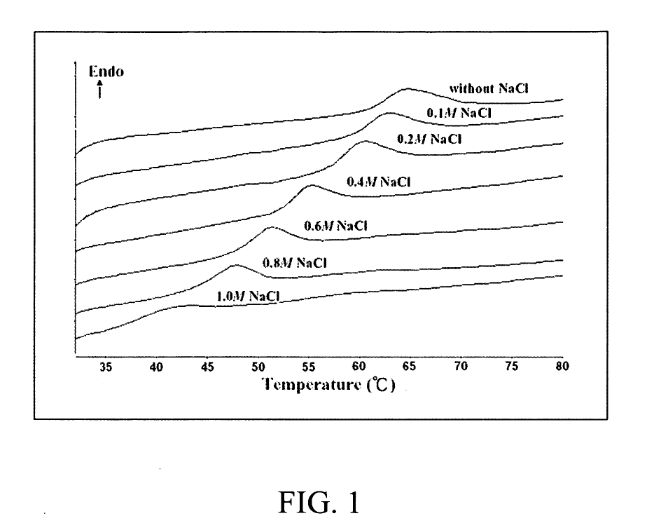

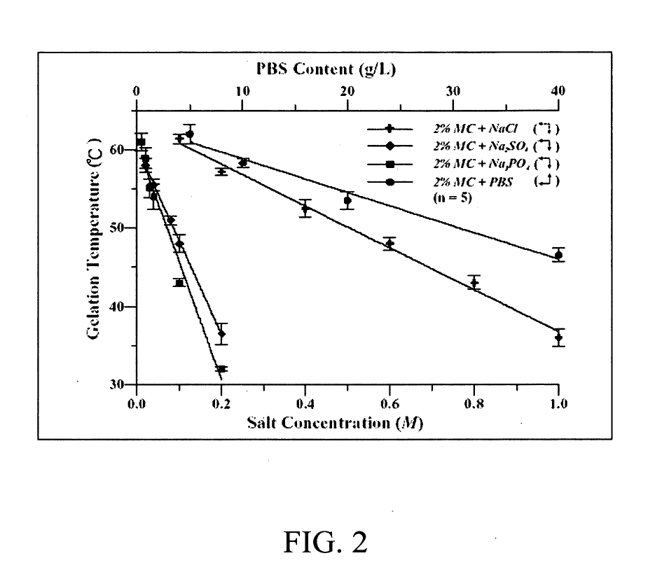

The salts blended in aqueous MC solutions played an important role in their physical sol-gel behavior. Examples of the DSC thermograms of aqueous MC solutions (2% by w/v) blended with distinct concentrations of NaCl are shown in FIG. 1. An endothermic peak was observed for each test sample in the heating process. With increasing the concentration of NaCl, the endothermic peak shifted to the left (p<0.05). This indicates that addition of NaCl in the aqueous MC solution led to its sol-gel transition occur at a lower temperature. Additionally, a higher concentration of NaCl used, a lower temperature in its sol-gel transition was observed. This fact was also observed in the determination of the gelation temperature of each test sample by inverting its container (FIG. 2). As expected, the onset temperatures of the endothermic peaks of aqueous MC solutions observed in the DSC thermograms were lower than their corresponding gelation temperatures obtained by the inversion method, ranged approximately from 1° C. to 3° C. (Table 1).

Similar phenomena were observed when Na₂SO₄, Na₃PO₄, or PBS was blended into aqueous MC solutions (FIG. 2 and Table 1). Normally, an electrolyte (the salt blended) has a greater affinity for water than polymers resulting in removing water of hydration from the polymer and thus dehydrating or ‘salting out’ the polymer. The ability of an electrolyte to salt out a polymer from its solution generally follows the salt order in the lyotropic series. The cations follow the order Li⁺>Na⁺>K⁺>Mg²⁺>Ca²⁺>Ba²⁺, and more common anions follow the order PO₄³⁻>SO₄²⁻>tartrate>Cl⁻>NO₃⁻>Br⁻>I⁻>SCN⁻ (Int. J. Pharm. 1990; 99:233). Accordingly, more water molecules were removed from aqueous MC solutions when Na₂SO₄ or Na₃PO₄ was added in the polymeric hydrogel, resulting in a lower gelation temperature. As shown in FIG. 2 and Table 1, at the same concentration of the salt blended, generally, the gelation temperatures of aqueous MC solutions followed the order Na₃PO₄<Na₂SO₄<NaCl (p<0.05).

Effects of addition of PBS in aqueous MC solutions on the onset temperatures of the endothermic peaks observed in the DSC thermograms and their gelation temperatures were similar to those blended with NaCl, Na₂SO₄, or Na₃PO₄ (FIG. 2 and Table 1). It was reported that the effect of cations on salting-out polymers in solution is less significant than that of anions. Therefore, salting-out MC polymers from aqueous solutions blended with PBS was mainly caused by its constituent anions such as Cl⁻, HPO₄²⁻, or H₂PO⁴⁻.

TABLE 1
The onset temperatures (Tₒₙₛₑₜ) of the endothermic peaks of aqueous
methylcellulose solutions (2% by w/v) blended with distinct salts in varying
concentrations (Conc.) observed in the DSC thermograms and their gelation
temperatures (T_{gelation}) measured by an inversion method (n = 5).
NaCl

| | Conc. (M) | | | | | |
|---|---|---|---|---|---|---|
| | 0.1 | 0.2 | 0.4 | 0.6 | 0.8 | 1.0 |
| Tₒₙₛₑₜ | 59.0 ± 0.8 | 55.6 ± 0.3 | 52.0 ± 0.1 | 47.4 ± 0.3 | 42.3 ± 0.4 | 35.2 ± 0.3 |
| T_{gelation} | 61.4 ± 0.6 | 57.2 ± 0.4 | 52.5 ± 1.1 | 48.0 ± 0.8 | 43.0 ± 0.9 | 36.0 ± 1.1 |

Na₂SO₄

| | Conc. (M) | | | | |
|---|---|---|---|---|---|
| | 0.02 | 0.04 | 0.08 | 0.10 | 0.20 |
| Tₒₙₛₑₜ | 57.3 ± 0.2 | 54.8 ± 0.3 | 50.4 ± 0.4 | 47.4 ± 0.3 | 35.1 ± 0.3 |
| T_{gelation} | 58.0 ± 0.8 | 55.5 ± 0.7 | 51.0 ± 0.5 | 48.0 ± 1.1 | 36.5 ± 1.3 |

Na₃PO₄

| | Conc. (M) | | | | | |
|---|---|---|---|---|---|---|
| | 0.01 | 0.02 | 0.03 | 0.04 | 0.10 | 0.20 |
| Tₒₙₛₑₜ | 60.1 ± 0.5 | 58.4 ± 0.5 | 54.6 ± 0.5 | 53.4 ± 0.3 | 42 ± 0.4 | 30 ± 0.2 |
| T_{gelation} | 61.0 ± 1.1 | 58.9 ± 1.3 | 55.1 ± 1.1 | 54.0 ± 1.7 | 43 ± 1.1 | 32 ± 1.3 |

PBS

Conc. (g/L)

| | 5 | 10 | 20 | 30 |
|---|---|---|---|---|
| Tₒₙₛₑₜ | 57.5 ± 0.2 | 55.1 ± 0.5 | 52.4 ± 0.3 | 44.1 ± 0.2 |
| T_{gelation} | 62.0 ± 1.2 | 58.3 ± 0.5 | 53.5 ± 1.1 | 46.5 ± 0.9 |

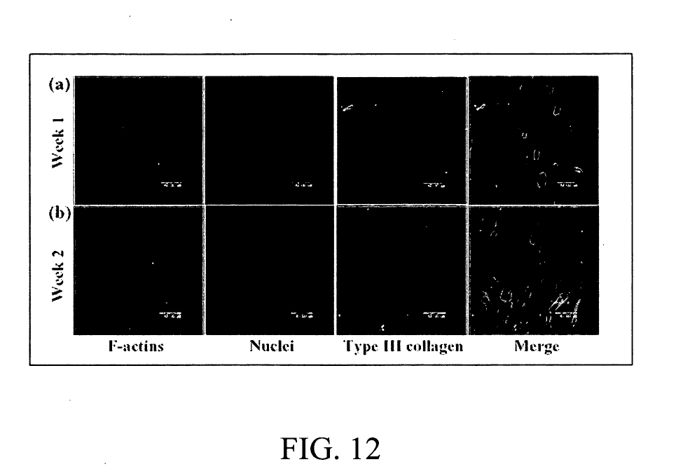

Results of the immunofluorescence images of the cell sheets grown on the MC/PBS/Collagen hydrogel for 1 and 2 weeks are shown in FIG. 12a and 12b, respectively. As shown, the F-actins and cell nuclei of the cultured cells (HFF) together with the secreted type III collagen were clearly identified. Type I collagen was also found in the study (data not shown). However, the labeled type I collagen may come from the originally coated bovine collagen or that secreted by the cultured cells. These results indicated the cultured cells could secrete their own ECM during culture. On the contrary, the originally coated bovine type I collagen may degrade gradually. It was reported that human skin fibroblasts could secrete collagenase as two proenzyme forms. These enzymes play an essential role in the maintenance of the ECM during tissue development and remodeling (Proc. Natl. Acad. Sci. U.S.A. 1986; 83:3756).

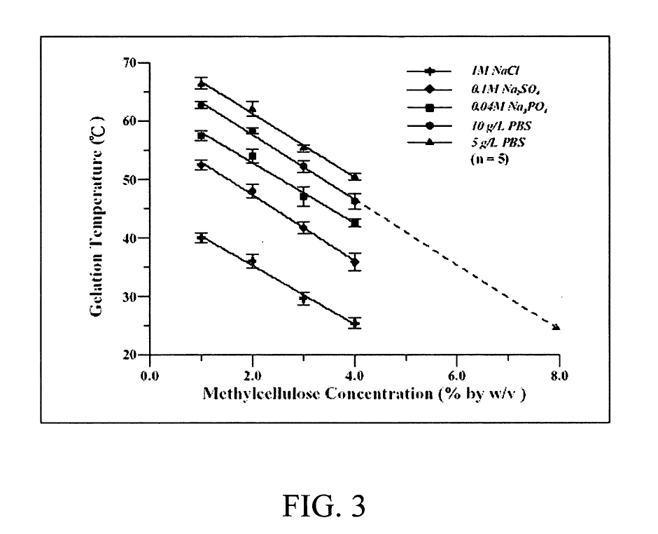

It was found that the concentration of MC in aqueous solution also played a significant role in its physical sol-gel behavior. As shown in FIG. 3, the gelation temperatures of aqueous MC solutions blended with distinct salts decreased approximately linearly with increasing the MC concentration. In the preparation of the aqueous MC solution, it was found that the solution was too viscous to be manipulated with when the MC concentration was greater than about 4% (by w/v). Therefore, no data were available when the concentration of MC was greater than this limit.

For the applications of cell culture, only those aqueous MC compositions that may form a gel (the MC hydrogel) at 37° C. were used to coat the TCPS dishes: 2% MC+1M NaCl; 2% MC+0.2M Na₂SO₄; 2% MC+0.2M Na₃PO₄ (FIG. 2); and 8% MC+10 g/L PBS. For the latter case, a 4% aqueous MC solution blended with 5 g/L PBS was used to coat the TCPS dish and subsequently dried in a laminar flow hood to remove 50% of its moisture content. Thus obtained MC hydrogel had a gelation temperature of about 25° C. (extrapolated from FIG. 3). As shown in FIG. 3, the gelation temperature of a 4% MC solution blended with PBS was significantly greater than 37° C. Additionally, as mentioned above, the aqueous MC solution was too viscous to be manipulated with when its concentration was greater than about 4%. It was observed that this specific aqueous MC solution (8% MC+10 g/L PBS) underwent a sol-gel reversible transition upon heating or cooling at approximately 25° C.

### EXAMPLE NO. 8

### Stability of the Coated MC Hydrogel

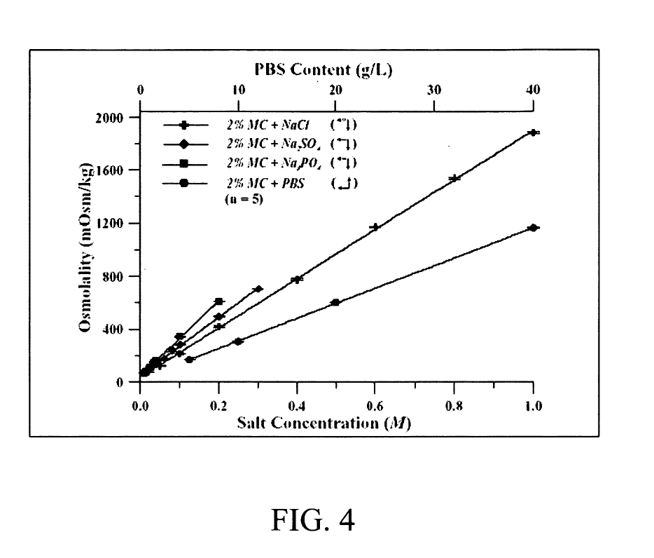

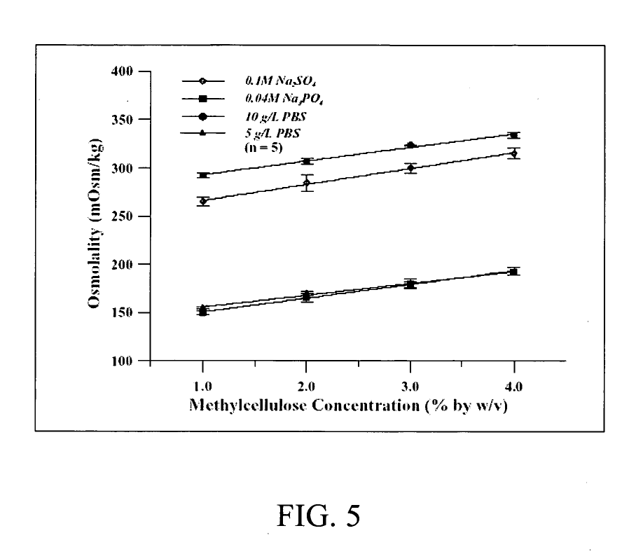

It is suggested that the MC hydrogels coated on TCPS dishes may be swelled and gradually disintegrated when loaded with the cell culture media due to the differences in osmotic pressure between the two. It was found that the osmolalities of aqueous MC solutions, used to prepare the MC hydrogels, increased nearly linearly with increasing the concentrations of the salt blended and MC (FIG. 4 and FIG. 5).

To evaluate the stability of the coated MC hydrogels, a PBS solution (10 g/L) with an osmolality of 280±10 mOsm/kg at 37° C., in simulating that of the cell culture media, was loaded on the coated TCPS dishes. The osmolality of the cell culture media is normally maintained at 290±30 mOsm/kg. An uncoated TCPS dish loaded with the same PBS solution was used as a control. Changes in osmolality of the loaded PBS solution with time were monitored by an osmometer.

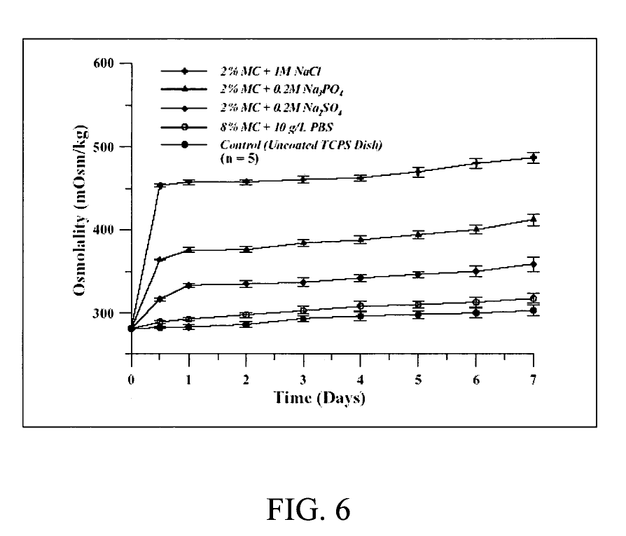

As compared to the uncoated control group, the osmolalities of the loaded PBS solutions increased significantly within 1 day (>325 mOsm/Kg) for the MC hydrogels blended with NaCl, Na₂SO₄, or Na₃PO₄ (p<0.05, FIG. 6). This observation might be attributed to the differences in osmolality between these MC hydrogels (>500 mOsm/kg, FIG. 4) and the originally loaded PBS solutions (about 280 mOsm/kg), and thus caused a significant amount of water from the loaded PBS solutions diffusing into the MC hydrogels. This leads to a significant increase in osmolality for the loaded PBS solutions together with a noticeable swelling and gradual disintegration of the MC hydrogels.

In contrast, the osmotic pressure of the PBS solution (10 g/L) loaded on the MC hydrogel blended with PBS (10 g/L) only increased slightly as compared to the uncoated control group (FIG. 6). Additionally, the MC hydrogel coated on the TCPS dish stayed intact throughout the entire course of the experiment. The aforementioned results indicated that the MC hydrogel blended with PBS (8% by w/v MC+10 g/L PBS) was more suitable for cell cultures than those blended with NaCl, Na₂SO₄, or Na₃PO₄, and thus was chosen for the study (the MC/PBS hydrogel).

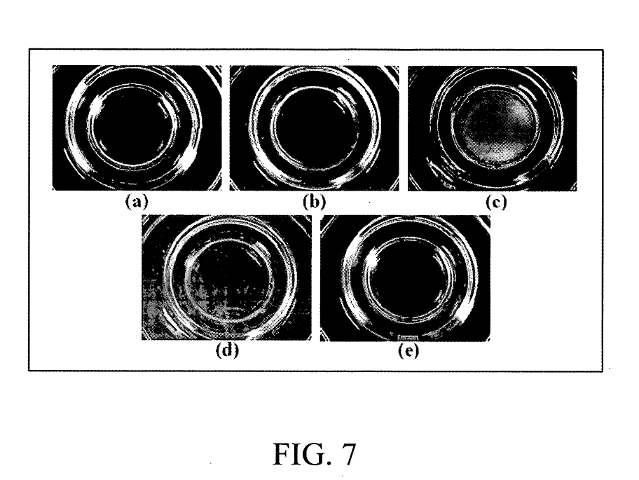

As shown in FIG. 7a, the MC/PBS hydrogel at 20° C. was a clear viscous solution. At 37° C., the clear solution starts to become opaque (FIG. 7b). The transition of sol-gel was continuous with time. At about 30 minutes later, a gel-network structure began to form (FIG. 7c). It was found that this hydrogel was thermoreversible. Back at 20° C., the opaque gel gradually became a clear viscous solution again (FIGS. 7d and 7e).

### EXAMPLE NO. 9

### Cell Culture on the Surface of the MC Hydrogel

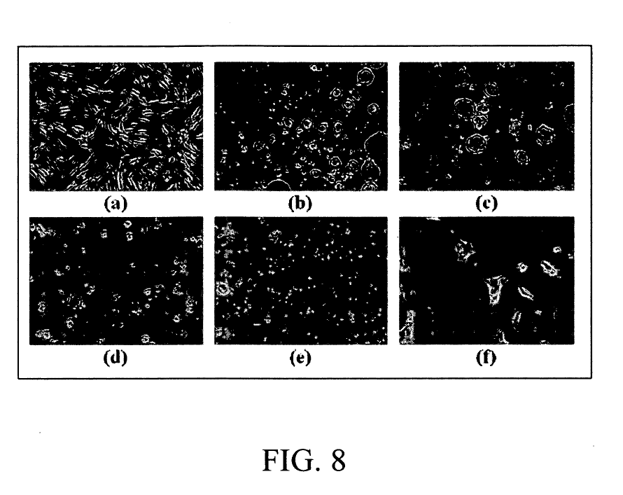

FIGS. 8a to 8f shows photomicrographs of cells (human foreskin fibroblasts, HFF) cultured on the surface of an uncoated TCPS dish (the control group) and those coated with the MC hydrogels blended with distinct slats for 1 day, respectively. As shown, the seeded cells attached very well on the surface of the uncoated TCPS dish (FIG. 8a). However, cells did not attach at all on the surfaces of the MC hydrogels blended with NaCl, Na₂SO₄, or Na₃PO₄ and mainly suspended in the culture media in the form of aggregates (FIGS. 8b-8d). In contrast, a few cells were found to attach on the surface of the MC/PBS hydrogel and the others remained to suspend in the culture media (FIGS. 8e and 8f).

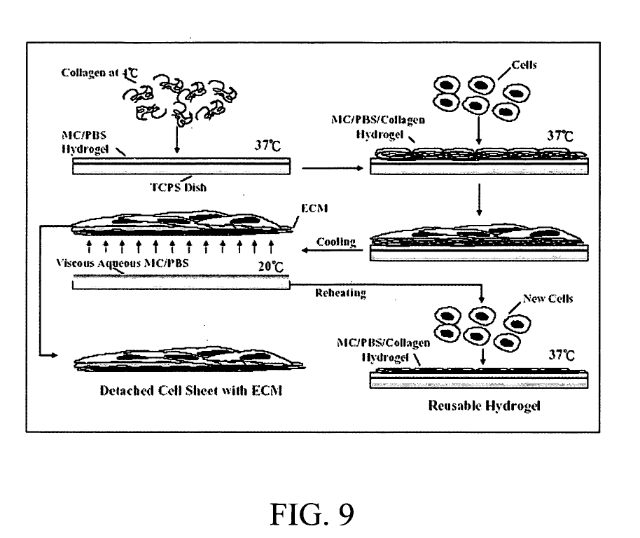

To improve cell attachments, a neutral aqueous bovine type I collagen at 4° C. was evenly spread on the TCPS dish coated with the MC/PBS hydrogel at 37° C. (FIG. 9). It was reported that under the influence of increasing temperature, collagen molecules self-assemble into a gel network. Thermal triggering of collagen gelation was demonstrated at a temperature as low as 20° C. and at a concentration as low as 0.1 mg/ml. Thus a thin layer of bovine type I collagen was formed on the surface of the MC/PBS hydrogel gradually (the MC/PBS/Collagen hydrogel, FIG. 9).

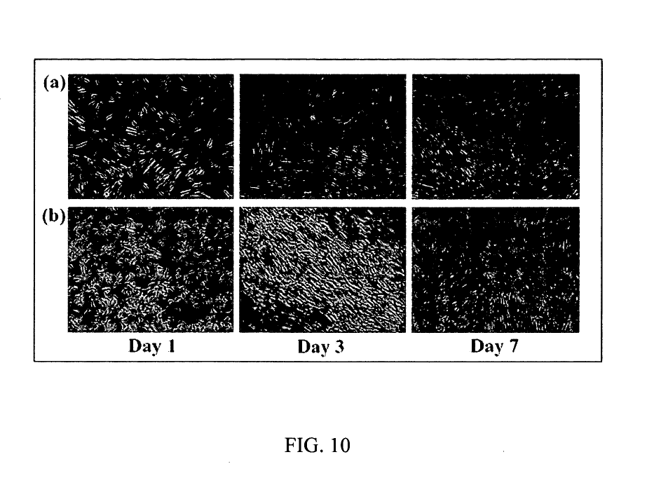

FIGS. 10a to 10i presents photomicrographs of cells cultured on an uncoated TCPS dish and that coated with the MC/PBS/Collagen hydrogel for 1, 3, and 7 days, respectively. Results of their relative-cell-activities of test-to-control evaluated by the MTT assay are shown in Table 2. As shown, after coating with the bovine type I collagen, cell attachments and proliferations were significantly improved as compared to those observed on the surface of the MC/PBS hydrogel (FIGS. 8e and 8f). The results obtained in the MTT assay demonstrated that the cells cultured on the surface of the MC/PBS/Collagen hydrogel had an even better activity than those cultured on the uncoated TCPS dish (p<0.05). Collagen is known to have the capacity to regulate cell behaviors such as adhesion, spreading, proliferation, and migration and thus has been used extensively to enhance cell-material interactions for both in vivo and in vitro applications.

TABLE 2
Results of the relative-cell-activities of test-to-control obtained in the
MTT assay for the cells cultured on an uncoated TCPS dish
(Uncoated Dish) and the TCPS dish coated with the MC/PBS/Collagen
hydrogel (Coated Dish) for 1, 3, and 7 days, respectively (n = 5).

| Relative Cell | | | |
|---|---|---|---|
| Activity^{[a]} | Day 1 | Day 3 | Day 7 |
| Uncoated Dish | 100.0 ± 2.3% | 161.9 ± 9.4% | 203.0 ± 12.3% |
| Coated Dish | 159.1 ± 7.7% | 286.3 ± 13.5% | 339.9 ± 18.7% |

^{[a]}The cell activity of the cells cultured on the uncoated TCPS dish for 1 day was used as a control.

### EXAMPLE NO. 10

### Detachment of Cell Sheets

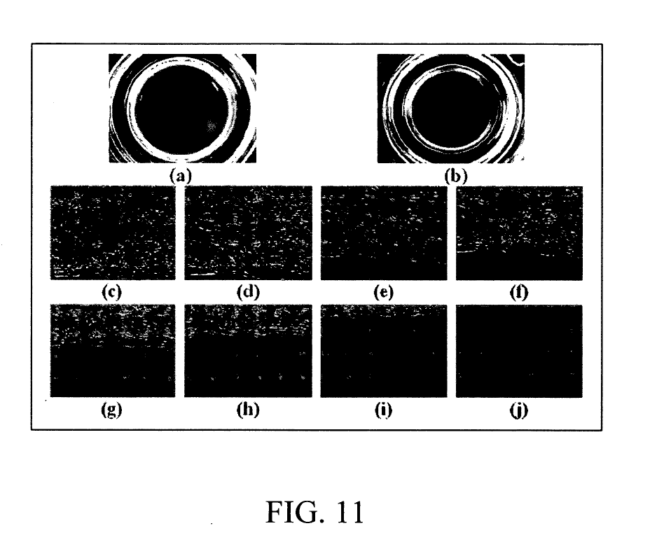

After cells reaching confluence, a continuous monolayer cell sheet formed on the surface of the MC/PBS/Collagen hydrogel (FIGS. 9 and 11a). When the grown cell sheet was placed outside of the incubator at 20° C., it detached gradually from the surface of the thermoreversible hydrogel spontaneously, in absence of any enzymes (e.g., trypsin/EDTA, FIGS. 9 and 11b-11j). It was observed that the grown cell sheet started to detach from its edge at about 2 minutes after cooling at 20° C. Detachment of the entire cell sheet was completed within 20 minutes (or within 10 minutes by shaking the TCPS dish gently with hand). With the same method, a large size of living cell sheet, cultured on a coated 100-mm petri dish, can be readily obtained in our lab and may be utilized in the applications of tissue reconstructions. FIG. 11 shows photographs of (a) a grown cell sheet on the TCPS dish coated with the MC/PBS/Collagen hydrogel and (b) its detaching cell sheet. Photomicrographs of the detaching cell sheet with time (c) to (j).

For most types of cells, and especially for a connective-tissue cell, the opportunities for anchorage and attachment depend on the surrounding matrix, which is usually made by the cell itself. It is known that fibroblasts are dispersed in connective tissue throughout the body, where they secrete an extracellular matrix (ECM) that is rich in type I and/or type III collagen (Molecular Biology of The Cell, 4^{th} ed., Garland Science, New York 2002, Ch.22). In one embodiment, the detached cell sheet was fixed and immunostained with anti-type I or type III collagen and subsequently co-stained with phalloidin for F-actins and propidium iodide for nuclei acids.

### EXAMPLE NO. 11

### Applications of the Developed Technique

After harvesting the detached cell sheet, the remained viscous MC/PBS hydrogel can be reused subsequent to recoating a thin layer of type I collagen on its surface as described before (FIG. 9). Additionally, a multi-layer cell sheet can be obtained with one of the following two methods. For the first method, a double-layer cell sheet can be obtained by seeding new cells directly on top of the first grown cell sheet (without detaching it from the surface of the MC/PBS/Collagen hydrogel) and then culture until confluence (FIG. 10b). The same procedure can be repeated again to obtain a tri-layer cell sheet (FIG. 10c). The other method is to fold the detached cell sheet into multi layers and reculture it. The folded multi-layer cell sheet would then stick together between layers within 2 days and form an integrated multi-layer cell sheet (FIG. 10d).

Some aspects of the present invention provide a method of preparing a living cell sheet comprising: coating a thermoreversible hydrogel on a tissue culture dish, wherein the hydrogel comprises methylcellulose, and phosphate buffered saline; loading target living cells into the dish; incubating the dish for a predetermined duration; and removing the sheet from the dish. In one embodiment, the hydrogel further comprises collagen. In another embodiment, the hydrogel further comprises at least one growth factor. In another embodiment, the target living cells are mesenchymal stem cells and/or adult multipotent cells.

The aforementioned single-layer or multi-layer cell sheets may be used in the applications of tissue reconstructions or tissue regeneration. Cell sheet engineering is being developed as an alternative approach for tissue engineering. It may have the advantages of eliminating the use of biodegradable scaffolds and maintaining the cultured cell-cell and cell-ECM interactions.

MSC cell sheet may not be easily injected by a needle or catheter into a body (for example, into myocardial tissue, into breast tissue, into an orthopedic space, or the like) of the patient due to its thickness. In one embodiment, each cell sheet (either single-layer or multi-layer sheet) could be broken up to several sub-cellsheets or cut to strips that are sized and configured to be appropriately loaded in a delivery instrument, such as a needle, a syringe, a catheter with a lumen or a cannule. In one embodiment, the cell strip with living cells is loaded into a delivery instrument with its long axis of the cell strip being aligned axially within the axial cavity or lumen of the delivery instrument. This is particularly important to provide therapeutically sufficient amount of MSC to a defect tissue for tissue regeneration by holding the MSC for long enough time on a sub-cellsheet in place at the target tissue site. On the contrary, cells leak or mobilize undesirably under the current cell therapy by injecting cell slurry or cell solution to the target tissue.

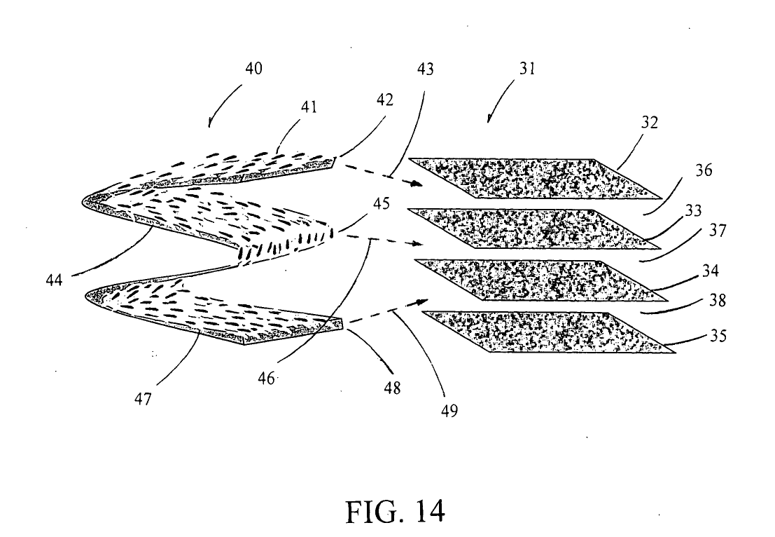

FIG. 14 shows a medical device comprising a support scaffold structure 31 of multiple layers (for example, some discrete layers 32, 33, 34, 35) that sandwich a single cell sheet 40 in between two adjacent scaffold layers, wherein the discrete layers 32, 33, 34, and 35 have a space 36, 37, and 38 between the respective layers as indicated. By way of illustration, a 3-layer living cell sheet 40 comprises layers 41, 44, and 47, whereby each sheet has its sheet edge 42, 45, 48 as indicated, respectively. In preparing a scaffold with living cells in a sandwich manner, the individual sheet edge of the cell sheet 40 is inserted into the space 36, 37, and 38, respectively. For example, the first sheet edge 42 moves toward the space 36 as shown in a dash-lined arrow 43. Similarly, the second sheet edge 45 moves toward the space 37 as shown in a dash-lined arrow 46 and the third sheet edge 48 moves toward the space 38 as shown in a dash-lined arrow 49. In an alternate embodiment, the three layers 41, 44, and 47 are three separate, non-connected living cell sheets.

The sandwiched scaffold may be sealed, secured, coupled, stapled, or sutured at at least one edge of the support scaffold structure to enable the composite medical device as a viable integral device or implant. By way of examples, the two adjacent layers with a living cell sheet in between may be sealed with fibrin glue, adhesives, pressure-sensitive adhesives, medical adhesive epoxy system, or cyanoacrylates. In one embodiment, the composite medical device of the invention with loaded living cells is sized and trimmed as a valvular leaflet used in a bioprosthetic tissue valve, as a pericardial patch for tissue regeneration, as a ligament/tendon substitute, as a breast insert for breast tissue regeneration, or as a wound dressing device. The sandwiched composite medical device has the benefits of the support scaffold (for example, an acellular tissue), such as good mechanical property, biocompatibility, and desired porous structure. The sandwiched composite medical device has the benefits of the living cell sheet (for example, multiple cell sheets), such as continuous cell-cell interaction, cell-ECM connection, and multiple cell stack in the composite device. In one embodiment, the support scaffold structure 31 is biodegradable.

In a co-pending patent application Ser. No. 10/408,176, filed Apr. 7, 2003, entitled “Acellular Biological Material Chemically Treated with Genipin”, entire contents of which are incorporated herein by reference, it is disclosed that the support scaffold is manufactured by a process comprising: removing cellular material from a nature tissue, wherein porosity of the nature tissue is increased at least 5%, the increase of porosity being adapted for promoting tissue regeneration. In one embodiment, increased porosity is provided by an acellularization process, an acid treatment process, or a base treatment process. In another embodiment, the manufacturing process for the support scaffold further comprises a step of crosslinking the nature tissue.

In some aspects, the single-layer living cell sheet passes through a laser-assisted cell identification and separation process, wherein a laser light with a cell-specific frequency passes through all cells on the cell sheet in a rotating or programmed manner to identify distinct cells to be preserved (for example, the myocardial stem cells in adipose derived tissue cells). For those non-specific cells or unwanted cells, a laser light with cell destroying energy is emitted to kill those cells. Thereafter, only desired cell type from the single-layer living cell sheet is obtained for cell differentiation and cell regeneration in a recipient. In one embodiment, fluorescence-coded cells or fluorescence light may be used for identifying distinct cells to be preserved to improve the purity of the living cell sheet.

By substituting the tissue culture dish with a 3-dimensional scaffold support element, hydrogel or partially gelled hydrogel of the invention may be loaded or coated onto the support element, followed by loading the target living cells and incubation. In one embodiment, the 3-D scaffold support element is biodegradable or bioresorbable so that the cells-loaded support element serves as an implant for in situ tissue regeneration in a recipient. The biodegradable material for the scaffold support element may be selected from a group consisting of chitosan, collagen, elastin, gelatin, fibrin glue, biological sealant, and combination thereof. The biodegradable material for the scaffold support element may be selected from a group consisting of polylactic acid (PLA), polyglycolic acid (PGA), poly (D,L-lactide-co-glycolide), polycaprolactone, and co-polymers thereof. The biodegradable material for the scaffold support element may be selected from a group consisting of polyhydroxy acids, polyalkanoates, polyanhydrides, polyphosphazenes, polyetheresters, polyesteramides, polyesters, and polyorthoesters.

In one exemplary illustration, hydrogel or partially gelled hydrogel of the invention may be loaded or coated onto the support scaffold element, followed by loading the target living cells and incubation. In one embodiment, the support scaffold element has multiple micropores that are connected to each other and in communication with the exterior surface openings. In another embodiment, the support scaffold element is a pericardial patch tissue, preferably an acellular patch tissue, and most preferably an acellular patch tissue with enlarged pores or increased porosity. U.S. Pat. No. 6,545,042, issued on Apr. 8, 2003, entire contents of which are incorporated herein by reference, discloses a method for promoting autogenous ingrowth of damaged or diseased tissue comprising a step of surgically repairing the damaged or diseased tissue by incorporating a tissue graft, wherein the tissue graft is formed from a segment of connective tissue protein after an acellularization process. In one embodiment, the cell-loaded tissue is sized and trimmed as a valvular leaflet used in a bioprosthetic tissue valve, as a pericardial patch for tissue regeneration, as a ligament/tendon substitute, as a breast insert for breast tissue regeneration, or as a wound dressing device.

Some aspects of the present invention provides a method of preparing a 3-D living cell construct comprising: coating or loading a thermoreversible hydrogel on a 3-D scaffold support element, wherein the hydrogel comprises methylcellulose, phosphate buffered saline, and collagen; loading target living cells onto the support element; incubating the support element for a predetermined duration. In one embodiment, the method further comprises a step of removing the construct from the support element.

Joint Repair or Reconstruction

Inflammation occurs at a joint, for example, associated with arthritis. An example of a joint disease is rheumatoid arthritis (RA) which involves inflammatory changes in the synovial membranes and articular structures as well as muscle atrophy and rarefaction of the bones, most commonly the small joints of the hands. Inflammation and thickening of the joint lining, called the synovium, can cause pain, stiffness, swelling, warmth, and redness. The affected joint may also lose its shape, resulting in loss of normal movement and, if uncontrolled, may cause destruction of the bones, deformity and, eventually, disability. In some individuals, RA can also affect other parts of the body, including the blood, lungs, skin and heart. One aspect of the invention provides delivering a living cell sheet with tissue regeneration capacity for reducing one or more of these adverse symptoms associated with RA.

The knee is a hingelike joint, formed where the thighbone, shinbone, and kneecap meet. The knee is supported by muscles and ligaments and lined with cartilage. Cartilage is a layer of smooth, soft tissue. It covers the ends of the thighbone and shinbone. For reference, U.S. Patent Application publication no. 2006/0029578, describes cartilage in terms of structure, function, development, and pathology in details. The cushioning cartilage can wear away over time. As it does, the knee becomes stiff and painful. Though a knee prosthesis can replace the painful joint, it is always better to regenerate and augment the cartilages with a medical device capable of restoring the cartilage functions by tissue regeneration, particularly the cells that can transform to chondrocytes and eventually to cartilage. One aspect of the invention provides a single cell sheet configured for transformable to chondrocytes at the worn cartilage for cartilage tissue regeneration. By “cartilage” is meant herein including articular cartilage, nose cartilage, ear cartilage, meniscus and avascular cartilage, patellar and spinal disk cartilage, and the like. The delivery means may be via less invasive needle injection or arthroscopic procedures.

A healthy knee joint bends easily. Movement of joints is enhanced by the smooth hyaline cartilage that covers the bone ends, by the synovial membrane that covers the hyaline cartilage and by the synovial fluid located between opposing articulating surfaces. Healthy cartilage absorbs stress and allows the bones to glide freely over each other. Joint fluid lubricates the cartilage surfaces, making movement even easier. A problem knee with worn, roughened cartilage no longer allows the joint to glide freely. Cartilage cracks or wears away due to usage, inflammation or injury. As more cartilage wears away, exposed bones rub together when the knee bends, causing pain. After implanting a living cell sheet, the cartilage is repaired and/or regenerated with new smooth surfaces and the bones can once again glide freely.

An exemplary apparatus for bone marrow collection, transport kit, implant kit and animal models that can be used in a method of the invention is described in U.S. Pat. No. 6,835,377 B2, which is well known to one ordinary skilled in the art and does not constitute a part of the current invention.

For osteoarthritis, rheumatoid arthritis, or fibromyalgia, the problems may occur to any joint, such as a finger joint, knee joint, hip joint, etc. Some aspects of the invention provide at least one living cell sheet as a medical implant for treating cartilage or condyles in arthritis or surface damage of cartilage or condyles. Within six to twelve weeks following implantation, the implant develops into fill thickness cartilage with complete bonding to the subchondral bone.

Chondrogenesis

This aspect focuses on the identification of molecules regulating mesenchymal stem cells during chondrogenic differentiation, including factors controlling the development of articular hyaline cartilage. To regenerate hyaline cartilage in osteoarthritis patients under a variety of clinical scenarios, it is important to develop a better understanding of the molecules that control the chondrogenic lineage progression of human mesenchymal stem cells. In vitro, it has been possible to culture human mesenchymal stem cells as “pellets” or aggregates under conditions that promote chondrogenesis in serum-free, defined media. This system permits the screening of molecules for chondrogenic potential in vitro. One aspect provides human mesenchymal cells in a single living cell sheet that promotes or enhances chondrogenesis in vivo and in situ.

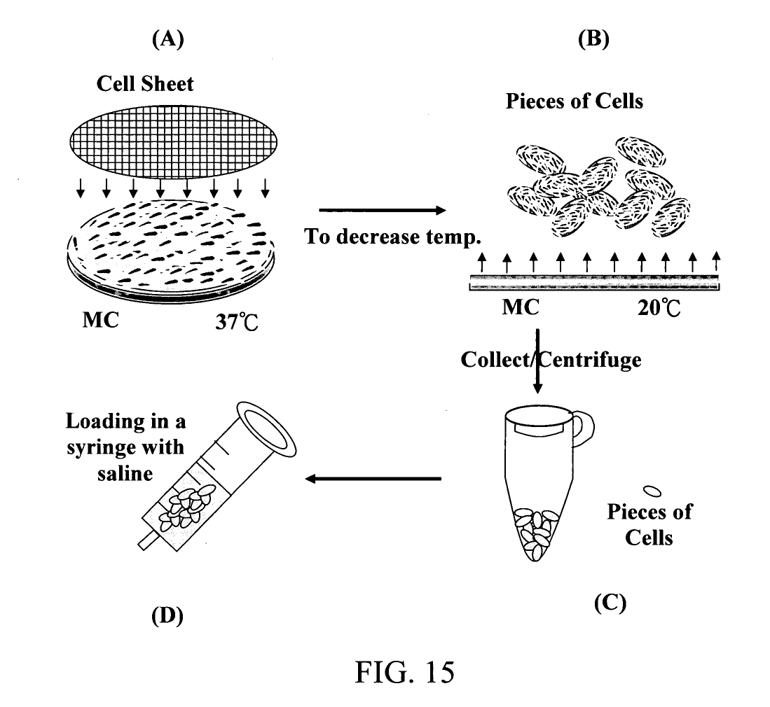

The cell sheet (see FIG. 15) provides biologically acceptable and mechanically stable surface structure suitable for genesis, growth and development of new non-calcified tissue. Other biologically active agents which can be utilized, especially for the reconstruction of articular cartilage, include but are not limited to transforming growth factor beta (TGF-beta) and basic fibroblast growth factor (bFGF).

Molecules that regulate gene expression, such as transcription factors and protein kinases, are useful for monitoring chondrogenesis in vitro, and make it possible to demonstrate, for each sheet or batch of cells, that the mesenchymal stem cells are maintained in an undifferentiated state and, once committed, the mesenchymal stem cell-derived progenitor cells are capable of progressing towards articular chondrocytes. Molecules that are secreted from the developing chondrocytes, such as extracellular matrix components and cytokines, are helpful in monitoring the chondrogenic process in vivo.

An exemplary biomatrix means that can be used in a method of the invention is described in co-pending U.S. patent application Ser. No. 11/287,865, filed Nov. 28, 2005, and entitled “pH sensitive hydrogel and drug delivery system”, which discloses a pharmaceutical composition for treating a joint of a patient, comprising: at least one bioactive agent; and a pH-sensitive hydrogel fluid, wherein the at least one bioactive agent is mixed with the hydrogel fluid, the hydrogel fluid solidifying at a physiological pH of the joint, preferably at a pH range of about 6.0 to 8.0, and most preferably at a pH range of about 7.0 to 7.8. In one embodiment, the bioactive agent is a living cell sheet, preferably a stem cell living cell sheet.

As disclosed, the pH sensitive or temperature sensitive hydrogel fluid may include: (1) a gel formulation that can be applied to osteochondral defects during arthroscopy; (2) an injectable cell-sheet suspension for delivery directly to the synovial space; and (3) a molded mesenchymal stem cell sheet-biomatrix product to re-surface joint surfaces in advanced cases. One aspect of the invention relates to the hydrogel fluid comprising N-akylated chitosan, wherein the chitosan is optionally crosslinked. Another aspect of the invention relates to the bioactive agent being an anti-inflammatory agent or an anti-infective agent. In one embodiment, the bioactive agent is selected from a group consisting of analgesics/antipyretics, antiasthamatics, antibiotics, antidepressants, antidiabetics, antifungal agents, antihypertensive agents, antineoplastics, antianxiety agents, immunosuppressive agents, antimigraine agents, sedatives/hypnotics, antipsychotic agents, antimanic agents, antiarrhythmics, antiarthritic agents, antigout agents, anticoagulants, thrombolytic agents, antifibrinolytic agents, antiplatelet agents and antibacterial agents, antiviral agents, and antimicrobials.

Some aspects of the invention relate to a pharmaceutical composition and a method for treating a joint defect in an animal, comprising administering to the animal stem cells, the stem cells being configured in a living cell sheet. In one embodiment, the method further comprises administering a biomatrix material. In one embodiment, the biomatrix material is a pH-sensitive hydrogel fluid, the hydrogel fluid solidifying at a physiological pH of the joint, preferably at a pH range of about 6.0 to 8.0, and most preferably at a pH-sensitive hydrogel fluid, the hydrogel fluid solidifying at a pH range of about 7.0 to 7.8.

FIGS. 9 and 15 shows cell sheet preparation and injection methods. First, as shown in FIG. 9 and Example No. 9, a cell sheet on MC is prepared by evenly spreading a neutral aqueous bovine type I collagen at 4° C. on the TCPS dish coated with the MC/PBS hydrogel at 37° C., followed by loading target cells onto the collagen suspension. After cells reaching confluence, a continuous monolayer cell sheet formed on the surface of the MC/PBS/Collagen hydrogel (FIGS. 9 and 11a). Second (see FIG. 15A), a cell sheet cutter is used to cut the whole cell sheet into pieces of cells configured for later injection delivery. When the grown cell sheet was placed outside of the incubator at 20° C. (see FIG. 15B), it detached gradually from the surface of the thermoreversible hydrogel spontaneously, in absence of any enzymes. Then pieces of cells at the pre-determined sizes and configuration are collected (see FIG. 15C) and loaded in a syringe (see FIG. 15D) along with saline or biomatrix of the invention for topical injection into a cavity or a joint.

Although the present invention has been described with reference to specific details of certain embodiments thereof, it is not intended that such details should be regarded as limitations upon the scope of the invention except as and to the extent that they are included in the accompanying claims. Many modifications and variations are possible in light of the above disclosure.

What is claimed is:

1. A method for treating a joint defect in an animal, comprising administering to said animal stem cells, said stem cells being configured in a living cell sheet.

2. The method according to claim 1, wherein the method further comprises administering a biomatrix material.

3. The method according to claim 2, wherein the biomatrix material is a pH-sensitive hydrogel fluid, said hydrogel fluid solidifying at a physiological pH of the joint.

4. The method according to claim 2, wherein the biomatrix material is a pH-sensitive hydrogel fluid, said hydrogel fluid solidifying at a pH range of about 6.0 to 8.0.

5. The method according to claim 2, wherein the biomatrix material is a pH-sensitive hydrogel fluid, said hydrogel fluid solidifying at a pH range of about 7.0 to 7.8.

6. The method according to claim 1, wherein the method further comprises administering a support scaffold having at least two layers, said living cell sheet being sandwiched in between said at least two layers.

7. The method according to claim 6, wherein the support scaffold is biodegradable.

8. The method according to claim 6, wherein the support scaffold is manufactured by a process comprising: removing cellular material from a nature tissue, wherein porosity of said nature tissue is increased at least 5%, the increase of porosity being adapted for promoting tissue regeneration.

9. The method according to claim 8, wherein increased porosity is provided by an acellularization process, an acid treatment process, a basic treatment process, or an enzyme treatment process.

10. The method according to claim 1, wherein the living cell sheet is manufactured by a process comprising:
coating a thermoreversible hydrogel on a tissue culture dish, wherein said hydrogel comprises methylcellulose and phosphate buffered saline;
loading said animal stem cells into said dish;
incubating said dish for a predetermined duration; and
removing said sheet from said dish.

11. The method according to claim 1, wherein the living cell sheet is characterized with promoting chondrogenesis in vivo.

12. The method according to claim 1, wherein the living cell sheet is sized and configured to be planar at about 100 microns in size.

13. The method according to claim 1, wherein the living cell sheet contains about at least 100 cells.

14. The method according to claim 1, wherein the method further comprises administering an anti-inflammatory agent or an anti-infective agent.

15. A method for treating cartilage defects in a patient, comprising delivering to said patient human cells in a sheet form, wherein said human cell sheet covers or contacts at least a portion of the defects.

16. The method according to claim 15, wherein the human cells are mesenchymal stem cells, marrow stromal cells, or chondrocytes.

17. The method according to claim 15, wherein the cartilage is selected from the group consisting of articular cartilage, nose cartilage, ear cartilage, meniscus, avascular cartilage, patellar, and spinal disk cartilage.

18. The method according to claim 15, wherein the method further comprises administering a biomatrix material.

19. The method according to claim 18, wherein the biomatrix material is a pH-sensitive hydrogel fluid, said hydrogel fluid solidifying at a physiological pH of the cartilage.

20. The method according to claim 15, wherein the living cell sheet is manufactured by a process comprising:
coating a thermoreversible hydrogel on a tissue culture dish, wherein said hydrogel comprises methylcellulose and phosphate buffered saline;
loading said human cells into said dish;
incubating said dish for a predetermined duration; and
removing said sheet from said dish.
